(19) United States
(12) Patent Application Publication
(10) Pub. No.: US 2022/0190453 A1
(43) Pub. Date: Jun. 16, 2022

(54) MICROWAVE SYSTEM AND METHOD

(71) Applicant: Emblation Limited, Alloa (GB)

(72) Inventors: Gary Beale, Alloa (GB); Eamon McErlean, Alloa (GB)

(73) Assignee: Emblation Limited, Alloa (GB)

(21) Appl. No.: 17/442,530

(22) PCT Filed: Mar. 25, 2020
(86) PCT No.: PCT/EP2020/058397

§ 371 Date: Sep. 23, 2021

(30) Foreign Application Priority Data
Mar. 25, 2019 (GB) 1904102.9

(51) Int. Cl. H01P 1/202; A61B 18/18; H01P 1/20

(52) U.S. Cl. CPC H01P 1/202; A61B 18/1815; A61B 2018/00577; A61B 2018/1892; A61B 2018/1823; H01P 1/2007

(57) ABSTRACT

A filtering device for use in a medical microwave delivery system is disclosed. The filtering device comprises a DC block and a microwave absorbing element. The microwave absorbing element is disposed around the DC block and/or around one or more elements within the DC block. Also disclosed is a medical microwave delivery system comprising a microwave generator system and the filtering device, wherein the filtering device is coupled to an output of the microwave generator system.

### FIELD

The present invention relates to a filtering device for suppressing electromagnetic interference (EMI) from a medical microwave delivery system, and a medical microwave delivery system used in the ablation of biological tissues in which such a filtering device is implemented, and an associated method of use and method of construction of said filtering device.

### BACKGROUND

Medical microwave delivery systems may be used in the ablation of biological tissues. In a medical microwave delivery system that is used for ablation, microwave energy may be delivered from a microwave energy generator, via a connecting cable, to a radiating applicator that transfers the microwave energy into the tissue. The radiating applicator may comprise a radiating element. The radiating element may be placed in contact with biological tissue of a patient, or surrounded by such tissue, or placed at a small distance from such tissue.

EN 60601 Medical Electrical Equipment and Systems is a European standard which defines a set of requirements for medical electrical equipment. Collateral standards such as IEC 60601-2-6:2012 define further requirements for microwave therapy equipment. Patient safety standards such as EN 60601 and related collateral standards may present a key design requirement for medical microwave delivery systems, which is that the medical microwave delivery system must be designed to ensure a sufficient level of electrical isolation between conductive and insulated patient-contacting elements and equipment earthed parts. A microwave energy generator of a medical microwave delivery system may therefore be isolated from the mains supply and chassis earth path of the medical microwave delivery system by means of a filtering device, to prevent contact with an earthed housing. The filtering device may comprise, for example, a medical grade isolating transformer and/or a DC block, which may provide an isolation barrier.

Various electromagnetic compatibility (EMC) regulations define requirements for microwave equipment, for example European standard EN55011 and FCC regulations for industrial, scientific and medical (ISM) equipment and radiated emissions. Electromagnetic compatibility regulations (IEC 60601-1-2:2014) set limits on electromagnetic radiation emission and immunity.

A further key design requirement for medical microwave equipment is the control of undesired electromagnetic radiation emission, such that electromagnetic interference (EMI) is not caused to nearby electronic equipment and such that the medical microwave delivery system is compliant with electromagnetic compatibility regulations. Emission of unwanted electromagnetic radiation that may be capable of causing electromagnetic interference may be referred to as EMI emission or EMI interference.

In some circumstances, undesired electromagnetic radiation emission may occur at a frequency different from frequencies of the fundamental delivery band (the band in which the medical microwave equipment is intended to deliver microwave energy). For example, in an exemplary system the fundamental delivery band may be 1 to 10 GHz but unwanted electromagnetic radiation emissions may manifest at other radio frequencies, for example in the 100 MHz to 300 MHz range.

A combination of a requirement for electrical isolation and the requirement for the control of undesired electromagnetic radiation emission poses a challenge to system designers.

It is an object of at least one embodiment of at least one aspect of the present invention to obviate or mitigate one or more problems or disadvantages of the prior art.

### SUMMARY OF INVENTION

According to a first aspect of the present invention there is provided a filtering device for use in a medical microwave delivery system, the filtering device comprising a DC block and a microwave absorbing element, the microwave absorbing element being disposed around the DC block, e.g. at least a portion of the DC block, and/or around one or more elements within the DC block.

Beneficially, the provision of a microwave absorbing element around the DC block may suppress, or attenuate, electromagnetic radiation emitted by the DC block. Thus, leakage of microwave energy, i.e. via radiation, from a system employing such a filtering device may be reduced to a level sufficient to comply with EMI requirements, and sufficient to permit other potentially sensitive devices and systems to operate safely within their associated EMC ratings.

Furthermore, use of such a filtering device in, for example, a medical microwave delivery system may permit adequate suppression of EMI whilst maintaining patient safety requirements for electrical isolation.

Beneficially, the microwave absorbing element may act to attenuate, filter or suppress microwave leakage from the DC block by absorption of the microwave energy, for example at or around a fundamental frequency and/or at or around one or more harmonic components, which may be converted to heat.

Thus, the DC block may be prevented from operating as an unintended radiating antenna.

A geometry and/or profile of the microwave absorbing element may be selected to provide suppression of electromagnetic radiation within a desired suppression band.

A material of the microwave absorbing element may be selected to provide suppression of electromagnetic radiation within a desired suppression band.

Beneficially, selection of an appropriate geometry and/or profile and/or material may help suppress or attenuate undesired radiation from the DC block, in particular over a frequency bands of interest. For example, a medical microwave delivery system providing a microwave signal with a fundamental frequency in the range of 1 to 10 GHz may not only exhibit undesired radiation at or around the fundamental frequency, but unwanted radiation may also occur at other frequencies, in particular at harmonics of the fundamental frequency. By employing the filtering device of the present invention, such unwanted frequency bands of radiation may be sufficiently suppressed or attenuated.

The desired suppression band may be between DC and 10 MHz. The desired suppression band may be between 10 MHz and 500 MHz. The desired suppression band may be between 100 MHz and 200 MHz. The desired suppression band may be between 100 MHz and 300 MHz.

The desired suppression band may be approximately 4.8 GHz to 5 GHz. This may correspond to a second harmonic of a frequency of 2.45 GHz. The frequency of 2.45 GHz may substantially correspond to an Industrial, Scientific and Medical (ISM) band, such as an ISM band from 2.4 GHz to 2.5 GHz.

References to an ISM frequency or an ISM band may correspond to frequencies generally reserved and/or used for Industrial, Scientific and Medical (ISM) purposes. For example, this may comprise ISM frequencies or bands defined by the International Telecommunication Union (ITU) Radio Regulations (article 5, 2012 edition). In particular, such ISM frequencies may correspond to, for example, a centre frequency of 2.45 GHz with a frequency range of 2.4 to 2.5 GHz, or a centre frequency of 5.8 GHz with a frequency range of 5.725 to 5.875 GHz.

The desired suppression band may be approximately 11.45 GHz to 11.7 GHz. This may correspond to a second harmonic of a frequency of 5.8 GHz. Similarly, the frequency of 5.8 GHz may substantially correspond to an ISM band, such as an ISM band from 5.725 GHz to 5.875 GHz.

The desired suppression band may be approximately 4.8 GHz to 11.7 GHz.

The desired suppression band may be approximately 4 GHz to approximately 12 GHz.

The desired suppression band may comprise a plurality of sub-bands. For example, the desired suppression band may comprise a sub-band at approximately 4.8 GHz to 5 GHz and a sub-band at approximately 11.45 GHz to 11.7 GHz.

For example, the microwave absorbing element may be configured to attenuate, filter, suppress, or effectively eliminate, microwave leakage from the DC block by absorption of the microwave energy at the first and/or second and/or other harmonic of any ISM frequency. In an example, the ISM frequency may substantially correspond to a frequency of 2.45 GHz. The ISM frequency may substantially correspond to a frequency within a band from 2.4 GHz to 2.5 GHz.

In an example, the ISM frequency may substantially correspond to a frequency of 5.8 GHz. The ISM frequency may substantially correspond to a frequency within a band from 5.725 GHz to 5.875 GHz.

It will be appreciated that the microwave absorbing element may additionally or alternatively be configured to attenuate, filter, suppress, or effectively eliminate, microwave leakage from the DC block by absorption of the microwave energy at a first and/or second and/or other harmonic of any non-ISM frequency, such as s frequency other than a frequency within a band from 2.4 GHz to 2.5 GHz and/or a frequency within a band from 5.725 GHz to 5.875 GHz.

Harmonics of the ISM or non-ISM frequency may occur at spot frequencies if the fundamental is a fixed frequency. That is, harmonics of a fixed ISM or non-ISM frequency may occur at frequencies that are an integer (whole-number) multiple of the fixed ISM or non-ISM.

If the ISM or non-ISM frequency is frequency modulated or swept, e.g. changes over time, then harmonics of the fixed ISM or non-ISM frequency may be spread, e.g. occur over multiple frequencies.

A geometry and/or a material of the microwave absorbing element may be selected to provide attenuation of a harmonic of an ISM frequency.

Filtering may be provided by means of the DC block. The DC block may be a filter. For example, the DC block may be adapted to filter a DC (direct current) signal or a DC component of a signal. Furthermore, the DC block may also be adapted to filter low frequency components of the signal. That is, the DC block may be configured to prevent, inhibit or otherwise reduce, attenuate or suppress a DC signal and/or a DC component of a signal and/or one or more low frequency components of a signal. Furthermore, the DC block may be configured to provide minimal or negligible filtering of high frequency components of the signal such as, for example, microwave frequency signal, i.e. signals with a frequency in the region of 300 MHz to 300 GHz. That is, the DC block may be configured not to prevent, inhibit or otherwise reduce, attenuate or suppress such high frequency signals or high frequency components of a signal. That is, the DC block may be configured relatively negligibly prevent, inhibit or otherwise reduce, attenuate or suppress such high frequency signals or high frequency components of a signal.

The microwave absorbing element may be formed as a unitary element.

The microwave absorbing element may have a substantially cylindrical shape or form. The microwave absorbing element may have a substantially tubular shape or form.

For example, the microwave absorbing element may be fitted around a dielectric housing of the DC block in a complete form, for example as a cylinder with a through bore along its central axis, through which the dielectric housing of the DC block may be inserted during construction.

The microwave absorbing element may comprise a plurality of sub-elements configurable to be arranged around the DC block.

For example, in an alternative embodiment, the microwave absorbing element may be comprised of one or more pieces which are fitted to the dielectric housing of the DC block. The one or more pieces may comprise, for example, a single piece of a flexible rubber absorber material such as MAST Technologies MR42-0008-01. The one or more pieces may be wrapped around the dielectric housing.

The DC block may be disposed within the microwave absorbing element.

The DC block may be at least partially enclosed by the microwave absorbing element.

The DC block may comprise a housing. The DC block may be disposed within the housing. The housing may comprise a dielectric material.

The DC block may be arranged along a central axis of the microwave absorbing element. The DC block may be arranged along a longitudinal axis of the microwave absorbing element.

The filtering device may comprise a sub-assembly. The sub-assembly may be disposed around the DC block.

Beneficially, such a construction readily lends itself to the use of microwave absorbing elements comprising alternative materials and formed or fitted using alternative processes. For example, a recess in the sub assembly, e.g. a recess between the sub-assembly and the DC block, may be easily filled with a viscous and/or curable material such as epoxy resin with a ferromagnetic or carbon filler. Such a viscous curable material may be the microwave absorbing element.

The microwave absorbing element may be provided as a curable material.

The microwave absorbing element may be contained within the sub-assembly.

The sub-assembly may be prevented from directly contacting the DC-block by the microwave absorbing element.

The sub-assembly may be prevented from directly contacting a housing surrounding the DC block by the microwave absorbing element.

The microwave absorbing element may have a geometry selected and/or may comprise a material selected to provide an attenuation of between 40 and 100 dB/cm at 10 GHz.

Preferably, the microwave absorbing element may have a geometry selected and/or may comprise a material selected to provide an attenuation of between 60 and 80 dB/cm at 10 GHz.

More preferably, the microwave absorbing element may have a geometry selected and/or may comprise a material selected to provide an attenuation of approximately 70 dB/cm at 10 GHz.

The microwave absorbing element may have a geometry selected and/or may comprise a material selected to provide an attenuation of at least 25 dB/cm at between 2 and 20 GHz.

Preferably, the microwave absorbing element may have a geometry selected and/or may comprise a material selected to provide an attenuation of at least 25 dB/cm at between 5 and 15 GHz.

More preferably, the microwave absorbing element may have a geometry selected and/or may comprise a material selected to provide an attenuation of at least 25 dB/cm at approximately 10 GHz.

More preferably, the microwave absorbing element may have a geometry selected and/or may comprise a material selected to provide an attenuation of at least 25 dB/cm at approximately 4.8 GHz to 5 GHz and/or an attenuation of at least 25 dB/cm at approximately 11.45 GHz to 11.7 GHz.

The microwave absorbing element may be or may comprise an electrically conductive material.

The microwave absorbing element may be or may comprise an elastomeric material.

The microwave absorbing element may be or may comprise a ferromagnetic material.

The microwave absorbing element may be embedded within the filtering device.

The filtering device may comprise an inner blocking path.

The inner blocking path may comprise conductive structures, such as coaxially arranged conductive structures, separated by an insulating material. The insulating material may be a dielectric material.

The outer blocking path may comprise conductive structures, such as coaxially arranged conductive structures, separated by an insulating material. The insulating material may be a dielectric material.

The outer blocking path may be arranged around the inner blocking path. The outer blocking path may be concentrically arranged around the inner blocking path.

In use, electromagnetic radiation may leak, e.g. be emitted, from the filtering device from a gap or gaps between the inner and/or outer conductive structures. The amount of electromagnetic radiation that leaks may be defined by a particular geometry (length and spacing) of the gap or gaps. The gap or gaps may comprise a dielectric material disposed within them. As such the gap or gaps may be capacitive gap(s).

The filtering device may be a DC block.

The microwave absorbing element may be disposed across the, or each, gap. During construction of the filtering device, microwave absorbing element may be disposed across the, or each, gap. Beneficially, the microwave absorbing element may be disposed in a location that does not interfere with an intended coupling of microwave energy from one side of the filtering device to the other. The microwave absorbing element may be arranged to not be disposed inside or across a gap between inner and/or outer conductive structures, thus avoiding negating any advantages of the filtering device.

The filtering device may comprise an insulated conducting element disposed within, around or adjacent a gap between the at least one inner conductive structures and/or the at least one outer conductive structures.

The filtering device may comprise a plurality of microwave absorbing elements.

The filtering device may comprise a first microwave absorbing element disposed around a first portion of the DC block.

The filtering device may comprise a second microwave absorbing element disposed around a second portion of the DC block.

The first microwave absorbing element and the second microwave absorbing element may be separated by a gap or space. That is, the first microwave absorbing element and the second microwave absorbing element may be physically separated. Beneficially, separation of the first and second microwave absorbing elements may avoid high voltage flashover (HIPOT).

The microwave absorbing element may be disposed upon the outer conductive structure adjacent to or before/after a junction between the outer conductive structure and a further outer conductive structure. Beneficially, such an arrangement may avoid creating a high potential bridge between the isolated outer conductors. Furthermore, to avoid high voltage flashover (HIPOT), the microwave absorbing element may be surrounded by insulating material and/or sufficiently and/or adequately spaced from spanning a gap between the isolated outer conductors to prevent HIPOT from occurring.

The microwave absorbing element may be disposed directly over and above or around the outer conductive structure before/after a junction between the conductive outer structure and a further conductive outer structure.

The microwave absorbing element may be disposed around an exterior of the filtering device.

An insulated conducting element may be disposed in the filtering device. The insulated conducting element may be disposed exterior to the outer conductive structures, e.g. in a capacitive gap exterior to the outer conductive structures. Beneficially, such an arrangement may attenuate the level of leakage of electromagnetic radiation at an undesired frequency. Advantageously, a microwave absorbing element manufactured from a ferromagnetic material, or at least a core of such a microwave absorbing element manufactured from a ferromagnetic material, may provide an electrical impedance that attenuates microwave energy leakage from the DC block, thereby reducing radiated signal levels from the DC block to a level which is acceptable to meet EMI and/or EMC requirements. Beneficially, an electrical isolation performance of the filtering device may be unchanged by the addition of the microwave absorbing element since a conductive path, and hence isolation, between an input connector and an output connector of the filtering device may be unaffected.

The microwave absorbing element may be or may comprise an epoxy resin.

The microwave absorbing element may be or may comprise carbon fillers.

The DC block may be a coaxial DC block.

The DC block may be an inner or an outer DC block. The DC block may be an inner and an outer DC block.

The DC block may comprise a capacitor in series with a central conductor.

The DC block may comprise a capacitor in series with an outer conductor.

The DC block may comprise an insulating material disposed around the outer conductor and/or the inner conductor.

Beneficially, use of a DC block with a microwave absorbing element being disposed around the DC block may provide a microwave transmission path with electrical isolation against DC and low frequency AC currents between input and output connectors of the DC block, while permitting adequate suppression of EMI. Such a DC block may comprise capacitive separate inner and/or outer coaxial conducting paths using one or more low loss dielectric materials, for example PTFE or Kapton.

The microwave absorbing element may be secured to the DC block, or the housing surrounding the DC block, by means of an adhesive.

The microwave absorbing element may be secured to the DC block, or the housing surrounding the DC block, by means of one or more retaining components or fasteners, such as one or more cable ties.

The microwave absorbing element may be secured to the DC block, or the housing surrounding the DC block, by means of a plastic housing and/or a polymer heat shrink.

The means of securing the microwave absorbing element to the DC block may be an electrically insulating means.

The plastic housing or polymer heat shrink may at least partially cover an exterior surface of the microwave absorbing element.

The means of securing the microwave absorbing element to the DC block, or the housing surrounding the DC block, may be arranged such that the means does not conductively connect an input connector to an output connector of the DC block.

The microwave absorbing element may be arranged such that it does not extend to or contact the input connector and/or the output connector of the DC block.

A gap, or gaps between the microwave absorbing element and the input connector and/or the output connector may comprise, or be filled with, a dielectric material.

Such a gap or gaps may be deliberately maintained to ensure that there is no electrically conductive path between connectors of the filtering device. In alternative embodiments, for example where a smaller gap is desired to reduce emissions, a dielectric material such as Kapton may be applied within the gap to increase insulative properties of the gap or gaps.

Where means of securing the microwave absorbing element to the DC block such as retaining components are used, these retaining components may be carefully designed and positioned so as not to compromise an electrical isolation between the input connector and the output connector of the filtering device. As such, it may be beneficial to provide any such means of securing the microwave absorbing element to the DC block as electrical insulators. The microwave absorbing element may be incorporated into the construction of the DC block, such as being placed as a tube of microwave absorbing material over a central insulator of the DC block before the filtering device receives its connectors.

The one or more elements within the DC block may comprise at least one inner and/or at least one outer conductive structure.

The filtering device may comprise an insulated conducting element disposed within, around or adjacent a gap between the at least one inner conductive structures and/or the at least one outer conductive structures.

According to a second aspect of the present invention, there is provided an electromagnetic interference (EMI) suppressor for use in a medical microwave delivery system, the EMI suppressor comprising a microwave absorbing material adapted to be disposed around a DC block.

According to a third aspect of the present invention, there is provided a use of a microwave absorbing material to suppress EMI from a DC block in a medical microwave delivery system.

According to a fourth aspect of the present invention, there is provide a method of construction of a filtering device according to the first aspect, the method comprising the steps of disposing a microwave absorbing element around a DC block and providing input and output connectors to the DC block.

The step of disposing a microwave absorbing element around a DC block may comprise disposing the microwave absorbing element within a housing surrounding the DC block.

According to a fifth aspect of the present invention there is provided a medical microwave delivery system comprising a microwave generator system and a filtering device according to the first aspect, wherein the filtering device is coupled to an output of the microwave generator system.

The system may be for use in ablation of biological tissue.

The microwave generator system may comprise a medical grade isolation transformer. The medical grade isolation transformer may be adapted to provide a power supply to the microwave generator system. The power supply may be isolated from a mains power supply.

The microwave generator system may be disposed within an earthed enclosure.

The medical grade isolation transformer may be disposed within an/the earthed enclosure.

The filtering device may be disposed within an/the earthed enclosure.

The system may comprise a coaxial cable. The coaxial cable may be configured to transfer microwave energy from the microwave generator system to a microwave delivery device.

The microwave delivery device may comprise a microwave applicator. The microwave applicator may be configured to transfer microwave energy to tissue of a patient.

The microwave applicator may be used to deliver the microwave energy to biological tissue of a patient. In an example embodiment, the microwave applicator may be used to perform ablation of biological tissue. In other embodiments, the microwave applicator may be used to supply microwave energy for other medical purposes.

Microwave energy may be delivered from the microwave generator system, via the filtering device and a connecting cable, such as the coaxial cable, to the microwave applicator.

The microwave applicator may comprise a monopolar electrode. The microwave applicator may comprise a radiating applicator that transfers microwave energy into tissue of a patient. The radiating applicator may comprise a radiating element. In use, the radiating element may be placed in contact with biological tissue of a patient. In use, the radiating element may be surrounded by such tissue, or placed at a small distance from such tissue.

The coaxial cable may be coupled to the output of the filtering device.

According to a sixth aspect of the present invention, there is provided an electromagnetic interference suppressor system for a medical microwave energy delivery system, comprising the fitment of a microwave absorbing material around a microwave DC block which is used to provide electrical isolation between a patient and the microwave generator system earthing path.

The microwave absorber material may be selected to provide a suitable attenuation across the desired frequency band for which suppression of EMI is required.

The microwave absorbing material may be manifested as a solid form with an internal through hole, for example but not limited to, a cylindrical form with hole along its central axis through which the DC block may be inserted.

The microwave absorbing material may be manifested as one or more pieces which are fitted around the DC block dielectric housing.

The microwave absorbing material may be manifested as a viscous curable material such as epoxy resin with a ferromagnetic or carbon filler within a sub-assembly secured to the DC block dielectric housing.

The microwave absorbing material may be manifested as a viscous curable material such as epoxy resin with a ferromagnetic or carbon filler within the DC block dielectric housing.

According to an seventh aspect of the present invention there is provided a DC block for a medical microwave delivery system, the DC block comprising a filtering component adapted to attenuate electromagnetic radiation within a desired suppression band.

The filtering component may comprise a shield. The shield may be a metallic shield.

At least a portion of the filtering component may be disposed around a DC blocking component of the DC block. The filtering component may form a substantially cylindrical shape or box-like shape.

The filtering component may be adapted to attenuate leakage at 4.8 GHz to 5 GHz by at least 25 dB/cm, and preferably by at least 50 dB/cm.

The filtering component may be adapted to attenuate leakage at 11.45 GHz to 11.7 GHz by at least 25 dB/cm, and preferably by at least 50 dB/cm.

The filtering component may be formed integral to the DC block. That is, the filtering component may form an integral component of the DC block.

According to an eighth aspect of the present invention there is provided a method of construction of a DC block according to the seventh aspect, the method comprising providing the filtering component as an integral component of the DC block.

It should be understood that the features defined above in accordance with any aspect of the present invention or below relating to any specific embodiment of the invention may be utilised, either alone or in combination with any other defined feature, in any other aspect or embodiment or to form a further aspect or embodiment of the invention.

### BRIEF DESCRIPTION OF DRAWINGS

These and other aspects of the present invention will now be described, by way of example only, with reference to the accompanying drawings, which are:

FIG. 1 a schematic of a filtering device according to an embodiment of the invention;

FIG. 2 a schematic of a medical microwave delivery system according to an embodiment of the invention;

FIG. 3a-b a photograph of a portion of a medical microwave delivery system without a DC block, and a corresponding view of a spectrum analysis of emitted radiation;

FIG. 4a-b a photograph of a portion of a medical microwave delivery system with a DC block, and a corresponding view of a spectrum analysis of emitted radiation;

FIG. 5a-b a photograph of a portion of a medical microwave delivery system with a filtering device according to an embodiment of the invention, and a corresponding view of a spectrum analysis of emitted radiation;

FIG. 6a-b schematics of cross-sections of filtering devices according to an embodiments of the invention;

FIG. 7a-b schematics of cross-section of filtering devices according to further embodiments of the invention; and

FIG. 8a-c schematics of cross-sections of filtering devices according to further embodiments of the invention.

### DETAILED DESCRIPTION OF DRAWINGS

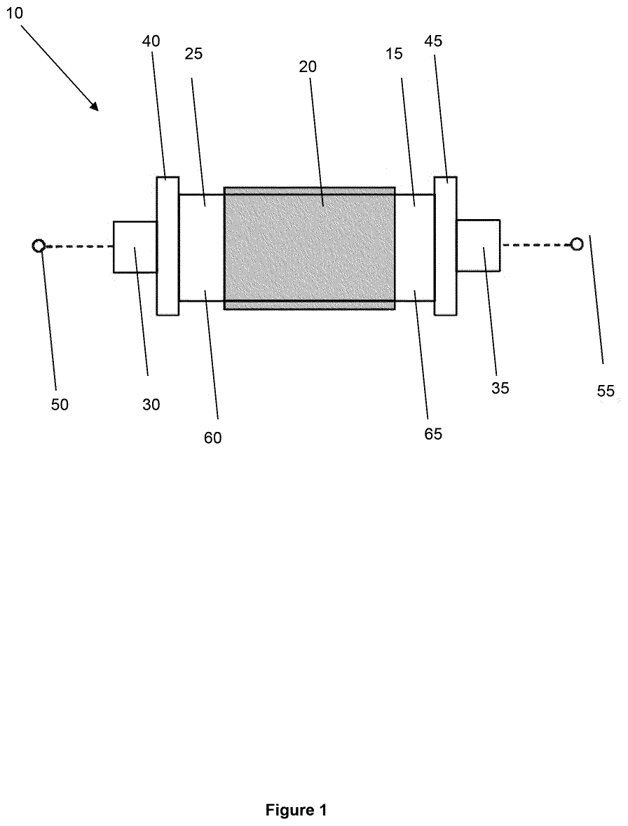

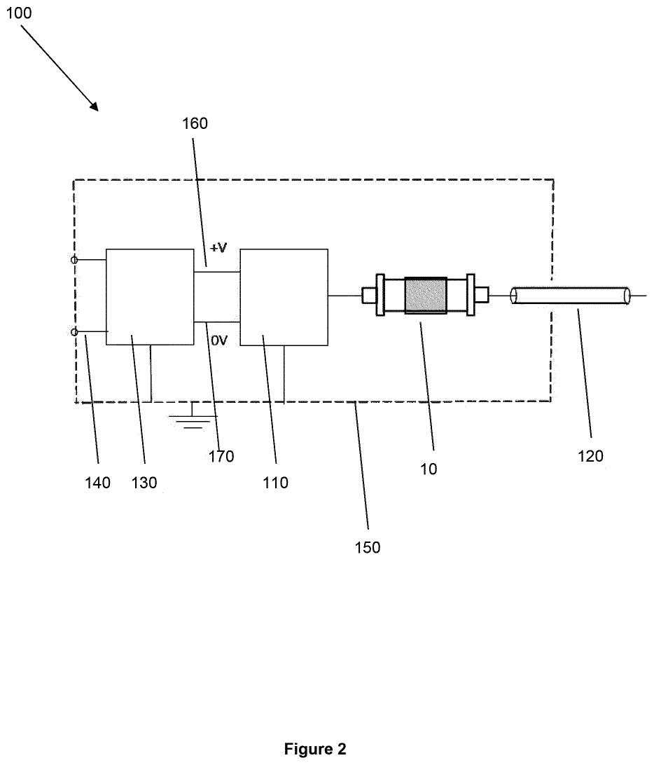

Referring firstly to FIG. 1, there is shown a filtering device generally denoted 10, for use in a medical microwave delivery system 100 (shown in FIG. 2). The filtering device 10 comprises a DC block 15 and a microwave absorbing element 20. The microwave absorbing element 20 is disposed around the DC block 15.

A geometry and or profile of the microwave absorbing element 20 is selected to provide suppression of electromagnetic radiation within a desired suppression band. In the example embodiment shown, the microwave absorbing element 20 is substantially cylindrical, and is disposed around the DC block 15. Furthermore, the microwave absorbing element 20 is shown as a unitary element, i.e. is of complete form. That is, the microwave absorbing element 20 is fitted around a dielectric housing 25 of the DC block 15 in a complete form, as a cylinder with a through bore along its central axis.

It will be understood that in other embodiments falling within the scope of the present invention, the microwave absorbing element 20 may comprise a plurality of sub-elements configurable to be arranged around the DC block 15. For example, in an alternative embodiment, the microwave absorbing element 20 may be comprised of one or more pieces which are fitted to the dielectric housing 25 of the DC block 15. The one or more pieces may comprise, for example, a single piece of a flexible rubber absorber material such as MAST Technologies MR42-0008-01.

The filtering device 10 comprises an input connector 30 and an output connector 35. The input connector 30 and the output connector 35 are coaxial connectors. Each connector 30, 35 is mounted on an associated connector mount 40, 45. The input connector 30 provides an electrically conductive path to an input to the DC block 15. In use, the input connector 30 may provide an electrically conductive path from a microwave source connection 50 to the input to the DC block 15. It will be appreciated that in other embodiments, the filtering device 10 may not comprises mounts that are distinct from the connectors, or such mounts may be integral to the connectors.

The output connector 35 provides an electrically conductive path to an output of the DC block 15. In use, the output connector 35 may provide an electrically conductive path from the output of the DC block 15 to a further coaxial cable 55, such as a patient cable connection.

In the example embodiment shown in FIG. 1, a gap 60 is shown between the microwave absorbing element 15 and the input connector mount 40. Similarly, a gap 65 is shown between the microwave absorbing element 15 and the output connector mount 45. The gaps 60, 65 ensure that the microwave absorbing element 15 does not provide electrical connectivity between the input connector 30 or input connector mount 40 and the output connector 35 or output connector mount 45.

It will be appreciated that in alternative embodiments falling within the scope of the present invention, one or both gaps 60, 65 may comprise, or be filled with, a material, such as a dielectric material or other insulating material. For example, a dielectric material such as Kapton may be applied within each gap 60, 65 to increase insulative properties of the gaps 60, 65.

Referring to FIG. 2, there is shown a medical microwave delivery system, generally denoted 100, and employing a filtering device 10 as shown in FIG. 1. The medical microwave delivery system 100 comprises a microwave generator system 110. The filtering device 10 is coupled to an output of the microwave generator system 110.

The medical microwave delivery system 100 comprises a microwave connecting cable 120 configured to transfer microwave energy from the microwave generator system 110 to a microwave delivery device (not shown), wherein the microwave connecting cable 120 is coupled to the output 55 of the filtering device 10. As such, the medical microwave delivery system 110 may be used in ablation of biological tissue.

The medical microwave delivery system 110 also comprises a power supply 130. The power supply 130 may be, for example, a medical grade isolation transformer. The power supply 130 is adapted to provide electrical power to the microwave generator system 110, wherein the electrical power provided to the microwave generator system 110 is isolated from a mains power supply 140. The power supply 130 may be a transformer, a power supply unit and/or may also include an ac/dc converter.

In the example embodiment shown, the power supply 130 provides a voltage supply 160 and a system ground 170 or 0V reference to the microwave generator system 110.

In medical applications requiring floating connectors the earthed enclosure 150 and the system ground 170 or 0V reference may be at different potentials due to the requirement to isolate the patient from the earth to prevent the risk of electrical shock.

The microwave generator system 110 and the power supply 130 are electrically connected to the earthed enclosure 130 (e.g. a chassis ground) which further assists in reducing overall system noise.

The medical microwave delivery system 110 and the power supply 130, which is a medical grade isolation transformer in the example embodiment shown are enclosed within an earthed enclosure 150.

Furthermore, the filtering device 10 is also shown as being enclosed within the earthed enclosure 150, although it will be appreciated that in alternative embodiments the filtering device 10 may be disposed outside the earthed enclosure 150. For example a coaxial cable may extend from an output of the microwave delivery system 110 to a filtering device 10 disposed outside the earthed enclosure 150.

In the example embodiment shown the microwave connecting cable 120 coupled to the output 55 of the filtering device 10 extends from within the earthed enclosure 150 to outside the earthed enclosure 150.

Such an arrangement, in particular the use of a DC block 15 in the filtering device 10, ensures that any conductive patient contacting parts of the system 100, for example a component of the microwave delivery device (not shown) are electrically isolated from the earthed enclosure 150 and from the mains supply 140.

In some embodiments of the invention, the microwave connecting cable 120 is a coaxial cable.

In an embodiment of the invention microwave connecting cable 120 may be of a single length, e.g. of unitary form, and may extend from the system 100 to an intended recipient device or target. In an alternative embodiment, a first microwave connecting cable (not shown) may extend within the enclosure 150 to a wall-mounted microwave coaxial connector arrangement (not shown), such as an SMP, BMA or SMA connector supplied by Amphenol or M/A-Com. A second microwave connecting cable (not shown) may then be connected to this connection externally to the enclosure 150.

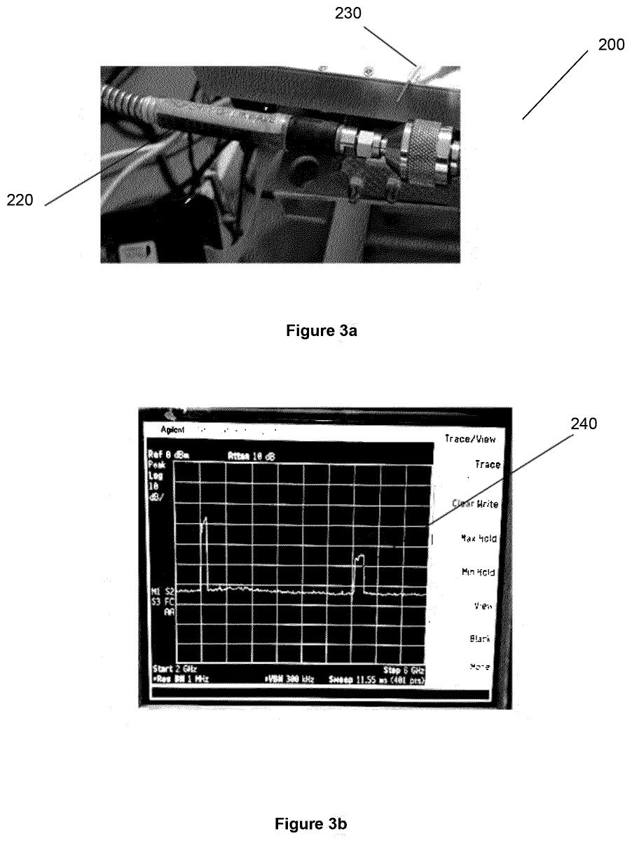

Referring to FIG. 3a there is shown a photograph of a medical microwave delivery system, generally denoted 200. The medical microwave delivery system 200 is connected to a microwave connecting cable 220 configured to transfer microwave energy from the medical microwave delivery system 200 to a microwave delivery device (not shown). Notably, the microwave connecting cable 220 is coupled directly to an output of the medical microwave delivery system 200. There is no filtering device according to the present invention present in this system 200.

A probe 230 is used to measure EMI from the microwave connecting cable 220 at approximately a point where the microwave connecting cable 220 connects to the medical microwave delivery system 200.

Referring now to FIG. 3b, there is shown a photograph of an oscilloscope display showing a spectral analysis of the EMI measured by the probe 230. A second harmonic 240 of a fundamental frequency of a microwave signal transmitted via the microwave connecting cable 220 is shown to be in the range of approximately 4.8 to 5 GHz.

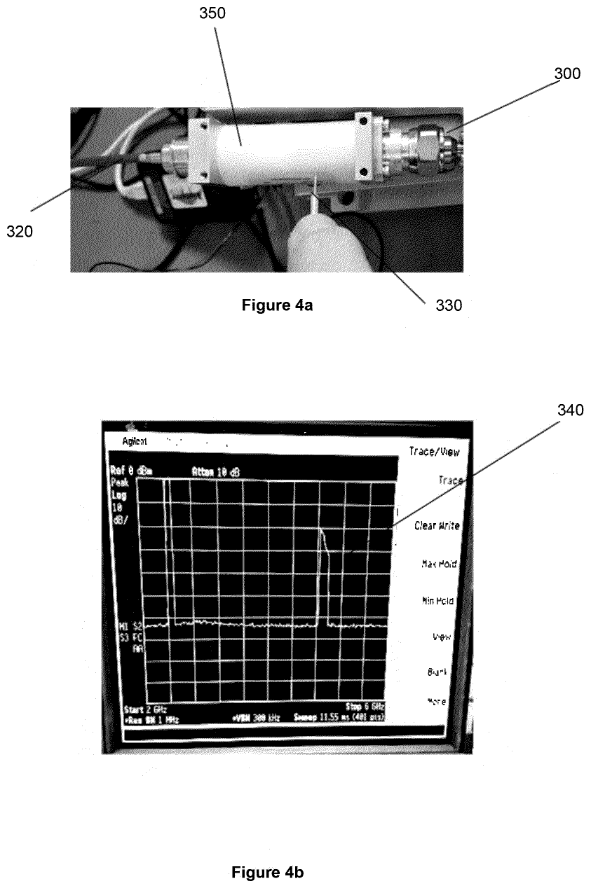

Turning now to FIG. 4a there is shown a further photograph of a medical microwave delivery system, generally denoted 300. The medical microwave delivery system 300 is connected to a microwave connecting cable 320 configured to transfer microwave energy from the medical microwave delivery system 300 to a microwave delivery device (not shown). However, in contrast to the medical microwave delivery system, generally denoted 200 of FIG. 3a, the medical microwave delivery system 300 of FIG. 4a is connected to the microwave connecting cable 320 via a DC block 350.

A probe 330 is used to measure EMI from the DC block 350. Referring now to FIG. 4b, there is shown a photograph of an oscilloscope display showing a spectral analysis of the EMI measured by the probe 330.

By comparing FIGS. 4b and 3b, it can be seen that the amplitude of the second harmonic 240, 340 over the range of approximately 4.8 to 5 GHz is increased by approximately 20 dB.

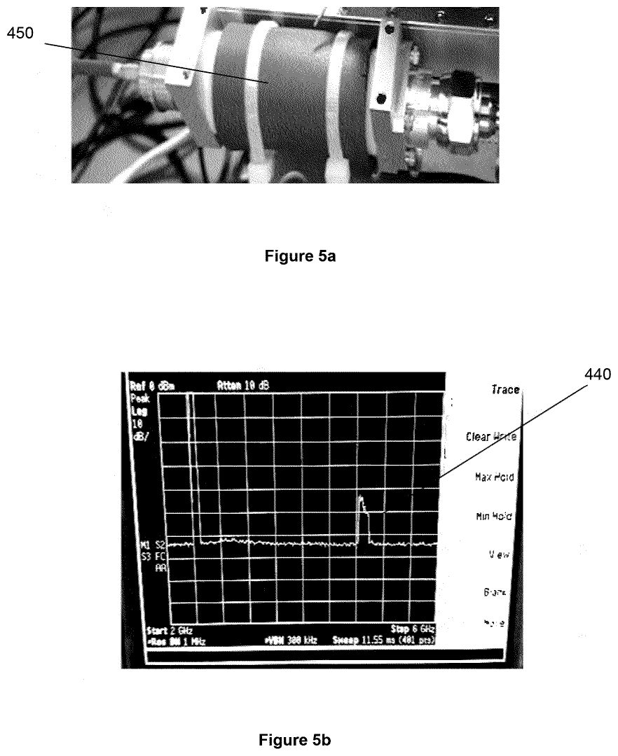

Turning now to FIG. 5a there is shown a similar arrangement as that of FIG. 4a. However, in this case a filtering device 450 according to the present invention is used in place of the DC block 350 of FIG. 4a.

FIG. 5b shows a further photograph of the oscilloscope display, this time showing a spectral analysis of the EMI measured by the probe 430 placed near the filtering device 450.

By comparing FIGS. 5b and 4b, it can be seen that the amplitude of the second harmonic 440, 340 over the range of approximately 4.8 to 5 GHz is decreased by approximately 20 dB. As such, the filtering device 450 of the present invention can be seen to significantly reduce EMI when compared to, for example, DC block 350 without a microwave absorbing element being disposed around the DC block 350.

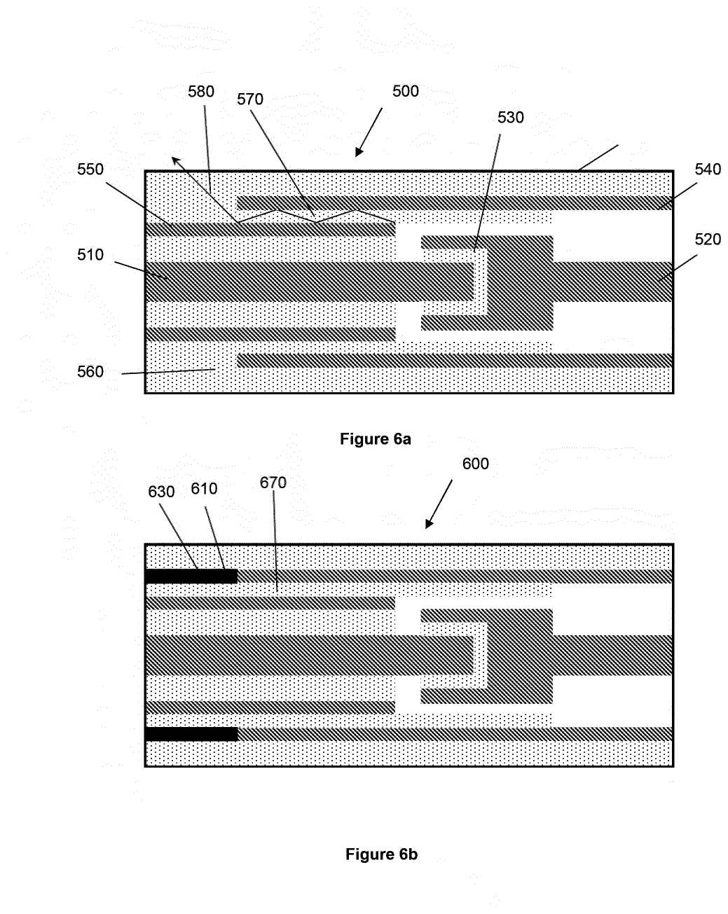

Referring to FIG. 6a there is shown a diagram of a cross-section of a filtering device. The filtering device is a coaxial DC block, generally denoted 500. The DC block 500 comprises an inner blocking path, formed by conductive structures 510, 520 separated by an insulating material 530 (for illustrative purposes the insulating material is represented by dot fill and the conductive structures 510, 520 are shown as hatched). The DC block 500 also comprises an outer blocking path, formed by outer conductive structures 540, 550 separated by an insulating material 560.

Electromagnetic radiation 580 can leak, and hence emit, from gaps 570 between the outer conductive structures 540, 550. The amount of electromagnetic radiation that is emitted relates to the particular geometry (length and spacing) of the capacitive gap.

Referring to FIG. 6b there is shown a diagram of a cross-section of a filtering device, generally denoted 600, according to an embodiment of the invention. The filtering device 600 is a DC block, and is similar to the DC block 500 of FIG. 6a. However, during construction, a microwave absorbing element 610 is disposed across the gap 670. Beneficially, the microwave absorbing element 630 is disposed in a location that does not interfere with the intended coupling of microwave energy from one side of the DC block to the other. In the example embodiment shown in FIG. 6b, the microwave absorbing element 610 is not disposed inside or across the gap 670, thus avoiding negating any advantages of the DC block component.

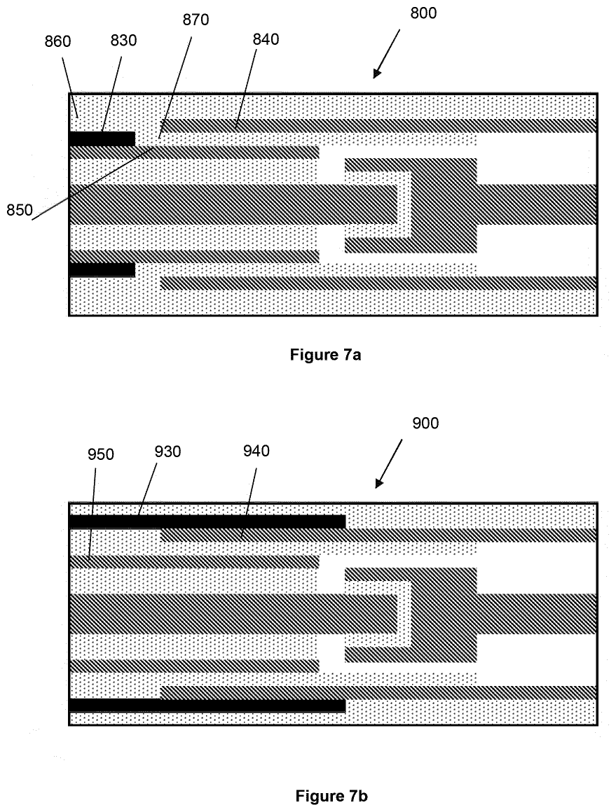

Referring to FIG. 7a there is shown a diagram of a cross-section of a further filtering device, generally denoted 800, according to an embodiment of the invention. In this embodiment, a microwave absorbing element 830 is disposed upon an outer conductor 850 before a junction with a further outer conductor 840. Beneficially, such an arrangement avoids creating a high potential bridge between the isolated outer conductors 840, 850. Furthermore, to avoid high voltage flashover (HIPOT), the microwave absorbing element 830 is surrounded by insulating material 860 and/or adequately spaced from spanning a gap 870 between the isolated outer conductors 840, 850 to prevent HIPOT from occurring.

In a further alternative embodiment shown in FIG. 7b, the microwave absorbing element 930 is disposed directly over and above the outer conductor 940 after the junction between the outer conductors 940, 950.

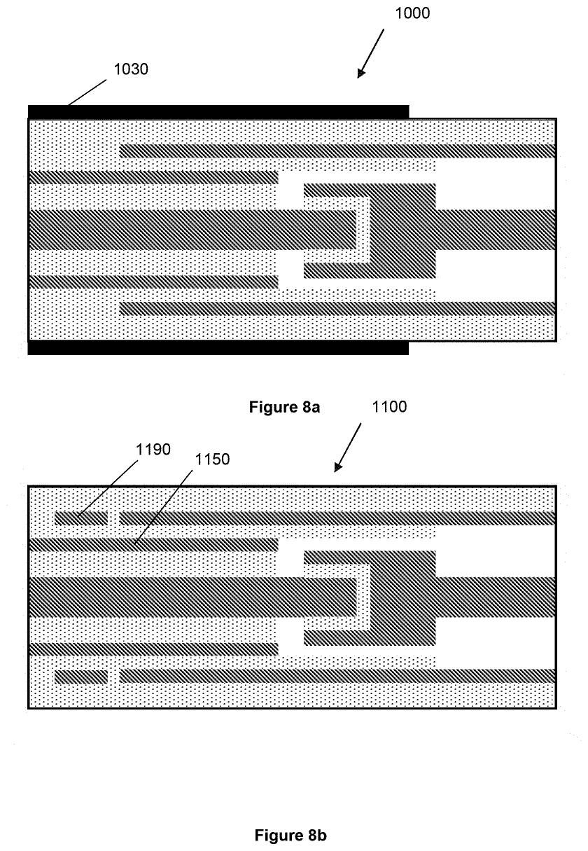

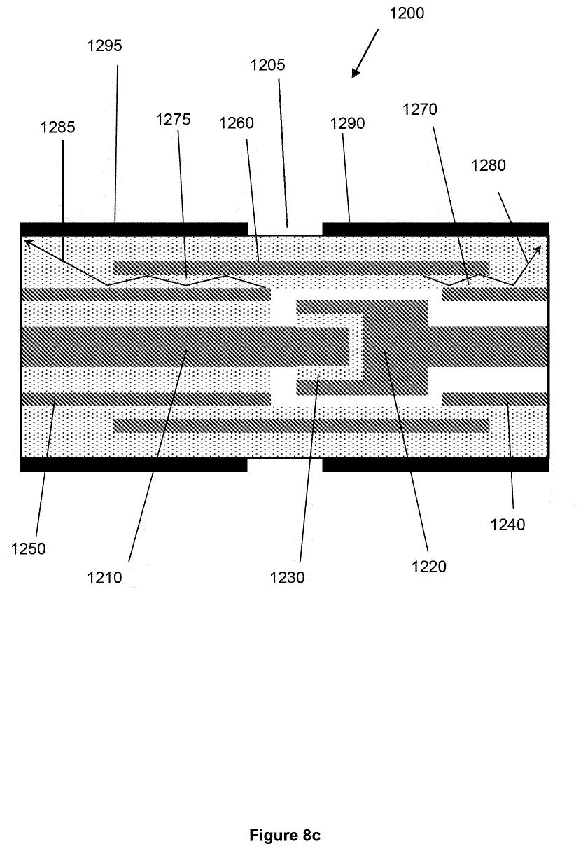

A further alternative embodiment is shown in FIG. 8a, wherein the microwave absorbing element 1030 is disposed around an exterior of the DC block 1000.

In yet a further alternative embodiment of a filtering device generally denoted 1100 and shown in FIG. 8b, an insulated conducting element 1190 is disposed in the DC block 1100. The insulated conducting element 1190 is disposed exterior to the outer conductive structures 1150, e.g. in a capacitive gap exterior to the outer conductive structures 1150. Beneficially, such an arrangement may attenuate the level of leakage of electromagnetic radiation at an undesired frequency. The insulated conducting element 1190 may be a microwave absorbing element.

FIG. 8c depicts a further embodiment of a filtering device, generally denoted 1200. The filtering device 1200 is a DC block, and comprises an inner blocking path, formed by conductive structures 1210, 1220 separated by an insulating material 1230.

The DC block 1200 also comprises an outer blocking path, formed by a first outer conductor 1240 and a second outer conductor 1250. The DC block also comprises a third outer conductor 1260. The third outer 1260 conductor is arranged such that it is separated from the first outer conductor 1240 and the second outer conductor 1250 by an insulating material. The third outer conductor 1260 is arranged such that it extends around a portion of the first outer conductor 1240. The third outer conductor is arranged such that it is extends around a portion of the second outer conductor 1250.

Electromagnetic radiation 1280 may leak, and hence emit, from a gap 1270 between the first outer conductive structure 1240 and the third outer conductive structure 1260. Similarly, Electromagnetic radiation 1285 may leak, and hence emit, from a gap 1275 between the second outer conductive structure 1250 and the third outer conductive structure 1260.

The amount of electromagnetic radiation that is emitted relates to a particular geometry (length and spacing) of the gaps 1270, 1275.

The filtering device 1200 of the embodiment of FIG. 8c comprises a first microwave absorbing element 1290 disposed around an exterior of a portion of the DC block 1200. In particular, the first microwave absorbing element 1290 is substantially disposed around the gap 1270 between the first outer conductive structure 1240 and the third outer conductive structure 1260. Beneficially, the first microwave absorbing element 1290 may absorb radiation 1280 that may be emitted from the gap 1270.

Similarly, the filtering device 1200 of the embodiment of FIG. 8c comprises a second microwave absorbing element 1295 disposed around an exterior of a portion of the DC block 1200. In particular, the second microwave absorbing element 1295 is substantially disposed around the gap 1275 between the second outer conductive structure 1250 and the third outer conductive structure 1260. Beneficially, the second microwave absorbing element 1295 may absorb radiation 1285 that may be emitted from the gap 1275.

The first microwave absorbing element 1290 and the second microwave absorbing element 1295 are provided as two separate microwave absorbing elements 1290, 1295. That is, a space 1205, e.g. a separation space or gap, is provided between the first microwave absorbing element 1290 and the second microwave absorbing element 1295. Beneficially, the provision of the space 1205 may maintain HIPOT isolation.

It should be understood that the features defined above in accordance with any aspect or any specific embodiment of the invention may be utilised, either alone or in combination with any other defined feature, in any other aspect or embodiment of the invention.

1. A filtering device for use in a medical microwave delivery system, the filtering device comprising a DC block and a microwave absorbing element, the microwave absorbing element being disposed at least one of around the DC block or around one or more elements within the DC block.

2-22. (canceled)

23. The filtering device according to claim 1, wherein at least one of a geometry or a material of the microwave absorbing element is selected to provide at least one of:
suppression of electromagnetic radiation within a desired suppression band; or
attenuation of a harmonic of an ISM frequency.

24. The filtering device according to claim 1, wherein the microwave absorbing element comprises a material selected to provide attenuation of at least one of between 40 and 100 dB/cm at 10 GHz, at least 25 dB/cm at between 4.8 GHz and 5 GHz, or at least 25 dB/cm at between 11.45 GHz and 11.7 GHz

25. The filtering device according to claim 1, wherein:
the microwave absorbing element is formed as a unitary element; or
the microwave absorbing element comprises a plurality of sub-elements configurable to be arranged around the DC block.

26. The filtering device according to claim 1, wherein at least one of:
the microwave absorbing element has a substantially cylindrical or tubular shape, the DC block being disposed at least one of within or at least partially enclosed by the microwave absorbing element,
the DC block is arranged along a central, longitudinal axis of the microwave absorbing element, the filtering device comprises a sub-assembly disposed around the DC block, or
the microwave absorbing element is provided as a curable material contained within the sub-assembly.

27. The filtering device according to claim 26, wherein the sub-assembly is prevented from at least one of directly contacting the DC-block, or a housing surrounding the DC block, by the microwave absorbing element.

28. The filtering device according to claim 1, wherein the microwave absorbing element is, or comprises, at least one of:
an electrically conductive material;
an elastomeric material;
a ferromagnetic material;
an epoxy resin; or
carbon fillers.

29. The filtering device according to claim 1, wherein the DC block is at least one of:
a coaxial DC block;
an inner DC block; or
an outer DC block.

30. The filtering device according to claim 1, wherein the microwave absorbing element is secured to the DC block, or the housing surrounding the DC block, by means of at least one of:
adhesive;
one or more retaining components or fasteners;
a cable tie;
a plastic housing;
polymer heat shrink; or
an electrically insulating means.

31. The filtering device according to claim 30,
wherein at least one of:
the plastic housing or polymer heat shrink at least partly covers an exterior surface of the microwave absorbing element; or
the means of securing the microwave absorbing element to the DC block, or the housing surrounding the DC block, does not conductively connect an input connector to an output connector of the DC block,
and wherein at least one of:
the microwave absorbing element does not extend to or contact the input connector and/or the output connector of the DC block; or
a gap between the microwave absorbing element and at least one of the input connector or the output connector comprises, or is filled with, a dielectric material.

32. The filtering device according to claim 1, wherein the one or more elements within the DC block comprises at least one of inner or outer conductive structure.

33. The filtering device according to claim 32, comprising an insulated conducting element disposed within, around or adjacent a gap between the at least one of the inner conductive structures or the outer conductive structures.

34. The filtering device according to claim 1, comprising a first microwave absorbing element disposed around a first portion of the DC block, and a second microwave absorbing element disposed around a second portion of the DC block, and optionally wherein the first microwave absorbing element and the second microwave absorbing element are separated by a gap.

35. An electromagnetic interference (EMI) suppressor for use in a medical microwave delivery system, the EMI suppressor comprising a microwave absorbing material adapted to be disposed around a DC block.

36. The method of construction of a filtering device according to claim 1, the method comprising the steps of:
disposing a microwave absorbing element around a DC block; and
providing input and output connectors to the DC block.

37. The method according to claim 36, wherein the disposing a microwave absorbing element around a DC block comprises disposing the microwave absorbing element within a housing surrounding the DC block.

38. A medical microwave delivery system comprising:
a microwave generator system; and
a filtering device according to claim 1,
wherein the filtering device is coupled to an output of the microwave generator system.

39. The medical microwave delivery system according to claim 38, wherein the system is for use in ablation of biological tissue.

40. The medical microwave delivery system according to claim 39, further comprising at least one of:
a coaxial cable configured to transfer microwave energy from the microwave generator system to a microwave delivery device, wherein the coaxial cable is coupled to the output of the filtering device; or
a medical grade isolation transformer adapted to provide a power supply to the microwave generator system, the power supply being isolated from a mains power supply, and optionally where the microwave generator system, the medical grade isolation transformer and the filtering device are disposed within an earthed enclosure.

41. A DC block for a medical microwave delivery system, the DC block comprising a filtering component adapted to attenuate electromagnetic radiation within a desired suppression band.

42. The DC block according to claim 41, wherein at least one of:
the filtering component comprises a metallic shield;
the filtering component is formed integral to the DC block; or
the filtering component is adapted to attenuate microwave radiation by at least 25 dB/cm over the suppression band,
wherein the suppression band is from at least one of 4.8 GHz to 5 GHz or 11.45 GHz to 11.7 GHz.

43. The method of construction of a DC block according to claim 41, the method comprising providing the filtering component as an integral component of the DC block.
